(19) United States
(12) Patent Application Publication
(10) Pub. No.: US 2025/0120767 A1
(43) Pub. Date: Apr. 17, 2025

(54) ASSESSING THE EFFECT OF A MEDICAL INSTRUMENT DURING A VASCULAR INTERVENTION

(71) Applicant: Siemens Healthineers AG, Forchheim (DE)

(72) Inventors: Marcus Pfister, Bubenreuth (DE); Philipp Roser, Erlangen (DE)

(21) Appl. No.: 18/914,253

(22) Filed: Oct. 13, 2024

(30) Foreign Application Priority Data
Oct. 13, 2023 (DE) 10 2023 210 068.1

(51) Int. Cl. A61B 34/10; G06T 7/00; G06T 7/62

(52) U.S. Cl. CPC A61B 34/10; G06T 7/0012; G06T 7/62; A61B 2034/104; A61B 2034/105; G06T 2207/10016; G06T 2207/10116; G06T 2207/30021; G06T 2207/30101

(57) ABSTRACT

For assessing the effect of a medical instrument during a vascular intervention, a sequence of images of a vessel section and of a first medical instrument that is situated in the vessel section is obtained. A deformation of the vessel section is ascertained based on the sequence of images. First mechanical instrument properties of the first medical instrument are obtained, and mechanical vessel properties of the vessel section are determined based on the first mechanical instrument properties and the deformation. Second mechanical instrument properties of a second medical instrument are obtained. An expected deformation of the vessel section during a vascular intervention on the vessel section using the second medical instrument is assessed based on the second mechanical instrument properties and the vessel properties.

This application claims the benefit of German Patent Application No. DE 10 2023 210 068.1, filed on Oct. 13, 2023, which is hereby incorporated by reference in its entirety.

### BACKGROUND

The present embodiments relate to assessing the effect of a medical instrument during a vascular intervention.

In vascular interventions (e.g., minimally invasive vascular interventions), treatments (e.g., placement of vessel supports, such as stents) or diagnoses (e.g., the detection of stenoses) are carried out by medical instruments introduced into the body. Such medical instruments may be, for example, the stents or vessel catheters (e.g., microcatheters). The navigation in the individual vessel branches or the placement of stents takes place by twisting and advancing a guide wire or catheter at the infeed point (e.g., the groin). This may be carried out, by way of example, using X-ray monitoring with angiography systems.

A plurality of differently shaped instruments exists for navigation as well as diagnosis and treatment in order to be able to optimally probe the individual vessel branches depending on the situation. As a rule, the individuals carrying out the treatment decide which instrument is suitable, or seems suitable, for which situation, on the basis of their experience. In the case of guide wires and vessel catheters, different instruments may be tried out; stents or other implants may be selected according to pre-operative planning.

These procedures are time-consuming, however. In addition, instruments that turn out to be unsuitable during testing have to be disposed of. Further, the patient is possibly administered an unnecessarily high volumes of contrast medium, and the exposure of patients and medical staff to radiation is increased unnecessarily.

The publication D. Roy et al., “Finite element analysis of abdominal aortic aneurysms: geometrical and structural reconstruction with application of an anisotropic material model,” IMA Journal of Applied Mathematics, 79, 5 (2014), pp. 1011-1026 describes simulation models for simulating the interaction of medical instruments with vascular structures based on pre-operative data.

The actual conditions (e.g., the actual vessel properties) may have changed during the course of the intervention compared to the pre-operative planning. This may result in sub-optimally suitable medical instruments being used (e.g., stents with sub-optimal dimensions are used or vessel catheters with sub-optimal rigidity, etc.).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a possibility for more accurately assessing the effect of a medical instrument on a vessel section during a vascular intervention is provided.

The present embodiments are based on the idea of ascertaining the actual deformation of a vessel section on the basis of images that show the vessel section and a first medical instrument in the vessel section, and based on this, of assessing the expected deformation of the vessel section during a potential vascular intervention with a second medical instrument.

According to one aspect of the present embodiments, a method for assessing the effect of a medical instrument (e.g., a second medical instrument) during a vascular intervention (e.g., a potential or pending vascular intervention) is provided. In this connection, a sequence of images is obtained. The images of the sequence of images (e.g., all images of the sequence of images) map a vessel section of a patient, and at least some of the images of the sequence of images show a first medical instrument arranged in the vessel section. A deformation of the vessel section is ascertained based on the sequence of images. First mechanical instrument properties of the first medical instrument are obtained. Mechanical vessel properties of the vessel section are determined based on the first mechanical instrument properties and the deformation. Second mechanical instrument properties of the second medical instrument are obtained. An expected deformation of the vessel section during the vascular intervention (e.g., the potential or pending vascular intervention on the vessel section using the second medical instrument, instead of using the first medical instrument) is assessed based on the second mechanical instrument properties and the mechanical vessel properties.

The method for assessing the effect of a medical instrument during a vascular intervention may therefore be understood as a method for assessing the expected deformation of the vessel section during the vascular intervention using the second medical instrument. Assessing the expected deformation may also be referred to as a prognosis of the expected deformation.

In different embodiments of the method, the method may be fully computer-implemented. Unless disclosed otherwise, all acts of such a computer-implemented method may be carried out by a data processing apparatus that has at least one computing unit. For example, the at least one computing unit is configured or adapted for carrying out the acts of the computer-implemented method. For this, the at least one computing unit may store, by way of example, a computer program that includes commands that, when executed by the at least one computing unit, prompt the at least one computing unit to execute the computer-implemented method.

A corresponding embodiment of the method that is not purely computer-implemented follows from each embodiment of the computer-implemented method in that corresponding method acts for generating the sequence of images (e.g., using an imaging modality, such as an X-ray-based imaging modality) are also incorporated.

The sequence of images corresponds to two or more successive images that are generated by an imaging modality while the first medical instrument is being introduced into the vessel section and/or after the first medical instrument has been introduced into the vessel section and/or the first medical instrument is being moved inside the vessel section. The introduction of the first medical instrument into the vessel section or the movement of the first medical instrument is not necessarily part of the inventive method or is not part of the method of the present embodiments. Generating the sequence of images may be part of the method in some embodiments and may take place before the method is carried out in other embodiments.

The vessel section is part of an anatomical vessel (e.g., a blood vessel, such as a vascular tree with a main vessel and one or more vessel branches).

The deformation may be a change in the shape of the vessel section (e.g., owing to an expansion in the radial direction and/or in the longitudinal direction and/or owing to a torsion, etc., such as of the main vessel). The deformation may correspond to a deformation during the generation of the sequence of images and/or in comparison with a rest state of the vessel section. The rest state may exist, by way of example, before the introduction of the first medical instrument or be obtained from pre-operative data.

Changes in position and/or location of corresponding parts of the vessel section may be determined from the sequence of images (e.g., after a corresponding segmentation of the vessel section or the vessel walls and possibly of the first medical instrument) in order to ascertain the deformation.

The mechanical instrument properties may include, by way of example, a geometric shape and/or a diameter and/or a length and/or a rigidity and/or a surface lubricity of the corresponding medical instrument. These variables may be determined before carrying out the method of the present embodiments. The determination of these variables (e.g., by gauging the corresponding medical instrument) may also be part of the method in some embodiments.

The mechanical vessel properties may include, by way of example, a rigidity and/or an elasticity (e.g., a volume elasticity coefficient) and/or a geometric shape (e.g., a diameter or a progression of the diameter along the longitudinal direction of the main vessel) of the vessel section. The mechanical vessel properties may, for example, also vary locally (e.g., along the longitudinal direction of the main vessel).

Reference should be made to the fact the mechanical vessel properties are not compulsively defined solely by the vessel section itself. It is also possible, by way of example, that tissue, such as internal organs, bones, muscle tissue, fat tissue, etc., surrounding the vessel section or adjoining the vessel section, affect the mechanical vessel properties.

Therefore, the expected deformation of the vessel section during use of the second medical instrument may be assessed via the method of the present embodiments with knowledge of the mechanical instrument properties of the first medical instrument and of the second medical instrument. This assessment is based on the actually established mechanical vessel properties of the vessel section and is therefore more accurate or more reliable than an assessment based solely on a simulation and/or pre-operative planning data.

The first medical instrument may be selected in accordance with known approaches (e.g., based on the experience of an individual carrying out the treatment and/or based on a simulation and/or on pre-operative planning data). The deformation that arises owing to the use of the first medical instrument therefore potentially deviates from the expected deformation owing to the use of the first medical instrument. This deviation may be attributed, for example, to based on a sub-optimal simulation model and/or a change in the mechanical vessel properties compared to pre-operative planning.

It is possible to decide whether the second medical instrument is suitable for the potential vascular intervention (e.g., whether it is more suitable than the first medical instrument or a third medical instrument) based on the assessed, expected deformation of the vessel section owing to the use of the second medical instrument. An optimally suitable medical instrument may thus be selected by assessing the expected deformation for different medical instruments. While the method of the present embodiments does not prevent the initially used medical instrument (e.g., the first medical instrument) from being sub-optimal, without the method of the present embodiments, it is, however, to be accepted, by way of example, that, even after a attempts, ultimately a sub-optimal medical instrument is being used.

Further, a simulation model for simulating the interaction of medical instruments with the vessel section, such as was used, by way of example, as the basis for the selection of the first medical device, may be updated and thus improved based on the assessed, expected deformation and/or based on the ascertained mechanical vessel properties. The thus improved simulation model may also be used, by way of example, for assessing the expected deformation of the vessel section due to use of the second medical instrument. The expected deformation may be assessed, for example, also by iterative application of the simulation model.

According to at least one embodiment, the expected deformation of the vessel section during the vascular intervention on the vessel section using the second medical instrument is assessed using a predefined simulation model as a function of the second mechanical instrument properties and the mechanical vessel properties.

This enables a reliable and accurate assessment of the expected deformation of the vessel section. The simulation model may be, by way of example, a model based on the finite element method.

According to at least one embodiment, physiological data of the patient, by way of example relating to the vessel section, is obtained. The mechanical vessel properties of the vessel section are determined based on the first mechanical instrument properties, the deformation of the vessel section, and the physiological data.

The physiological data may include, by way of example, properties of vessel walls of the vessel section. By way of example, the physiological data may qualitatively or quantitatively describe the existence of deposits, also referred to as plaque, on the vessel walls. The physiological data may also include, by way of example, a blood pressure and/or a blood flow rate in the vessel section. The physiological data may be determined, by way of example, intra-operatively.

According to at least one embodiment (e.g., before the sequence of images is generated and before the first medical instrument is introduced into the vessel section), a further expected deformation of the vessel section during a vascular intervention on the vessel section using the first medical instrument is assessed using the predefined simulation model as a function of first mechanical instrument properties and as a function of predefined initial vessel properties of the vessel section.

Therefore, it is possible to at least partially dispense with the recourse to empirical values of individuals carrying out the treatment. A great level of automation may consequently be achieved. The initial vessel properties may be ascertained, by way of example, based on standard values and/or pre-operative examinations.

According to at least one embodiment, the simulation model is updated as a function of the deformation ascertained based on the sequence of images and the further expected deformation.

The simulation model may consequently be improved for further implementations of the method of the present embodiments.

According to at least one embodiment, the simulation model is updated as a function of the mechanical vessel properties, determined based on the first mechanical instrument properties and the deformation, and the initial vessel properties.

The simulation model may be improved for further implementations of the method of the present embodiments as a result of this too.

According to at least one embodiment, the first medical instrument is selected from a predefined plurality of medical instruments as a function of the further expected deformation.

The plurality of medical instruments contains two or more medical instruments, including the first medical instrument and, by way of example, including the second medical instrument.

According to at least one embodiment, the expected deformation of the vessel section during a vascular intervention on the vessel section using the second medical instrument is assessed using the updated simulation model as a function of the second mechanical instrument properties and the mechanical vessel properties.

For example, the second medical instrument may be selected from the predefined plurality of medical instruments as a function of the assessed, expected deformation, or its suitability for the vascular intervention may be confirmed or evaluated.

In various embodiments, the assessed, expected deformation may be compared for this purpose with one or more predefined maximum values relating to the deformation of the vessel section. If the maximum values are overshot, the method may thus be repeated for a further medical instrument of the predefined plurality of medical instruments, where the second medical instrument in the method is then to be replaced by the other further medical instrument.

The procedure may also be iterative in this connection. By way of example, individual instrument properties may be purposefully varied. It is consequently also possible to discover, for example, which instrument properties exactly result in the maximum values being overshot. The selection of the medical instrument ultimately used may consequently be optimized further.

According to at least one embodiment, an item of user information that proposes the second medical instrument for use during a vascular intervention is output on the vessel section as a function of the expected deformation of the vessel section during a vascular intervention on the vessel section using the second medical instrument.

For example, the second medical instrument is proposed for use during the vascular intervention (e.g., is proposed only when the suitability of the second medical instrument for the vascular intervention has been confirmed, such as a result of the comparison of the assessed, expected deformation with one or more predefined maximum values).

The item of user information may be output by an output device (e.g., visually by a display and/or acoustically by a speech output by an audio output device). The individual carrying out the treatment may thus be more effectively supported, or the level of automation may be increased further.

According to at least one embodiment, the first medical instrument is a first stent or a first vessel implant, and/or the second medical instrument is a second stent or a second vessel implant.

The method of the present embodiments is particularly advantageous in this connection since the implantation of a potentially sub-optimally suitable stent may be prevented, and this would possibly adversely affect the desired effect of the stent.

According to at least one embodiment, the first mechanical instrument properties include a geometric shape and/or a diameter and/or a length and/or a rigidity and/or a surface lubricity of the first stent or of the first vessel implant.

According to at least one embodiment, the second mechanical instrument properties include a geometric shape and/or a diameter and/or a length and/or a rigidity and/or a surface lubricity of the second stent or of the second vessel implant.

The rigidity may be, by way of example, a longitudinal rigidity or a shear rigidity or a flexural rigidity or a torsional rigidity. The surface lubricity is, for example, a lubricity of an outer surface of the respective stent or vessel implant. The lubricity is given, by way of example, by an inverse coefficient of friction.

The diameter may be an internal diameter or an external diameter. The diameter may also be different at different points of the respective stent or vessel implant.

According to at least one embodiment, the first medical instrument is a first vessel catheter or a first guide wire, and/or the second medical instrument is a second vessel catheter or a second guide wire.

The method of the present embodiments is particularly advantageous in this connection since with sub-optimally selected vessel catheter or guide wire, it cannot be reliably provided that the purpose of the vessel catheter or guide wire (e.g., the positioning of a stent or a balloon dilatation) is optimally achieved.

According to at least one embodiment, the first mechanical instrument properties include a diameter and/or a geometric shape and/or a length and/or a rigidity and/or a surface lubricity of a tip or a shaft of the first vessel catheter or part of the tip or the shaft of the first vessel catheter.

According to at least one embodiment, the second mechanical instrument properties include a diameter and/or a geometric shape and/or a length and/or a rigidity and/or a surface lubricity of a tip or part of the tip of the second vessel catheter.

A vessel catheter therefore has, for example, a shaft and a tip attached to the shaft. The shaft and the tip may be made of different materials and have different geometric shapes. While the shaft is frequently a straight tube and is comparatively rigid, the tip may have different shapes such as loops, bends, etc. The tips may consequently reach, for example, different vessel branches to different extents. Different parts of the tip may also have different shapes.

The rigidity may be, for example, a longitudinal rigidity or a shear rigidity or a flexural rigidity or a torsional rigidity. The surface lubricity is, for example, a lubricity of an outer surface of the tip or of the shaft or of the respective part thereof.

The diameter may be an internal diameter or an external diameter. The diameter may also be different at different points of the respective shaft or the respective tip.

Reference should be made to the fact that the first medical instrument and the second medical instrument are not necessarily of the same type (e.g., are not necessarily both stents or both vessel catheters, etc.), although this is the case in some embodiments.

In other words, in some embodiments, the first medical instrument may be a stent, and the second medical instrument may be a vessel catheter, or vice versa.

Since the assessment of the expected deformation of the vessel section is based on the mechanical vessel properties and the second mechanical instrument properties, but does not directly depend on the type of the first medical instrument or the first mechanical instrument properties, the expected deformation may be assessed in such combinations too.

According to at least one embodiment, the sequence of images represents a movement of the first medical instrument.

For example, the first medical instrument is moved in the vessel section during image recording, and this results at least partially in the deformation of the vessel section. The deformation may thus be evaluated in detail, and the accuracy of the determination of the mechanical vessel properties, and thus the accuracy of the assessment of the expected deformation, may be increased.

According to at least one embodiment, the expected deformation is a deformation that is to be expected owing to an intentional movement of the second medical instrument in the vessel section.

According to at least one embodiment, the sequence of images is a sequence of X-ray images (e.g., of X-ray projection images).

According to a further aspect of the present embodiments, a data processing apparatus is disclosed. The data processing apparatus has at least one computing unit that is configured to carry out a method of the present embodiments for assessing the effect of a medical instrument during a vascular intervention.

A computing unit may, for example, be understood as a data processing device that includes a processing circuit. The computing unit may therefore process data for carrying out computing operations, for example. These possibly also include operations to carry out indicated instances of access to a data structure (e.g., a look-up table (LUT)).

The computing unit may include, for example, one or more computers, one or more microcontrollers, and/or one or more integrated circuits (e.g., one or more application-specific integrated circuit(s) (ASIC), one or more field programmable gate array(s) (FPGA), and/or one or more system(s) on a chip (SoC)). The computing unit may also include one or more processors (e.g., one or more microprocessors, one or more central processing units (CPU), one or more graphics processing units (GPU), and/or one or more signal processors, such as one or more digital signal processors (DSP)). The computing unit may also include a physical or a virtual group of computers or other of the units.

In different example embodiments, the computing unit includes one or more hardware and/or software interfaces and/or one or more memory units.

A memory unit may be configured as a volatile data memory (e.g., as a dynamic random access memory (DRAM)) or static random access memory (SRAM) or as a non-volatile data memory (e.g., as a read-only memory (ROM)), as a programmable read-only memory (PROM), as an erasable programmable read-only memory (EPROM), as an electrically erasable programmable read-only memory (EEPROM), as a Flash memory or Flash EEPROM, as a ferroelectric random access memory (FRAM), as a magnetoresistive random access memory (MRAM), or as a phase-change random access memory (PCRAM).

According to a further aspect of the present embodiments, an imaging system is disclosed that has a data processing apparatus of the present embodiments and an imaging modality that is configured to generate the sequence of images.

According to at least one embodiment, the imaging modality is an X-ray imaging modality with an X-ray source and an X-ray detector.

According to at least one embodiment, the X-ray imaging modality is configured as a C-arm device.

According to a further aspect of the present embodiments, a computer program with commands is provided. When the commands are executed by a data processing apparatus (e.g., a data processing apparatus of the present embodiments), the commands prompt the data processing apparatus to carry out a method of the present embodiments.

The commands may be in the form of program code, by way of example. The program code may be provided, by way of example, as binary code or Assembler and/or as a source code of a programming language (e.g., C), and/or as a program script (e.g., Python).

According to a further aspect of the present embodiments, a computer-readable storage medium is disclosed that stores a computer program of the present embodiments.

The computer program and the computer-readable storage medium are in each case computer program products with the commands.

Further features and combinations of features of the present embodiments may be found in the figures and their description as well as in the claims. For example, further embodiments of do not necessarily have to include all features of one of the claims. Further embodiments may have features or combinations of features that are not mentioned in the claims.

The present embodiments will be explained in more detail below using specific embodiments and associated schematic drawings. Same or functionally same elements may be provided with the same reference numerals in the figures. The description of same or functionally same elements is possibly not necessarily repeated with respect to different figures.

### BRIEF DESCRIPTION OF THE DRAWINGS

FIG. 1 shows a schematic representation of an example embodiment of an imaging system;

FIG. 2 shows a schematic flowchart of an example embodiment of a method for assessing the effect of a medical instrument during a vascular intervention;

FIG. 3 shows a schematic representation of medical instruments;

FIG. 4 shows a schematic representation of different parts of a medical instrument from FIG. 3;

FIG. 5 shows schematic representations of a medical instrument in a vessel section; and

FIG. 6 shows a further schematic representation of a medical instrument in a vessel section.

### DETAILED DESCRIPTION

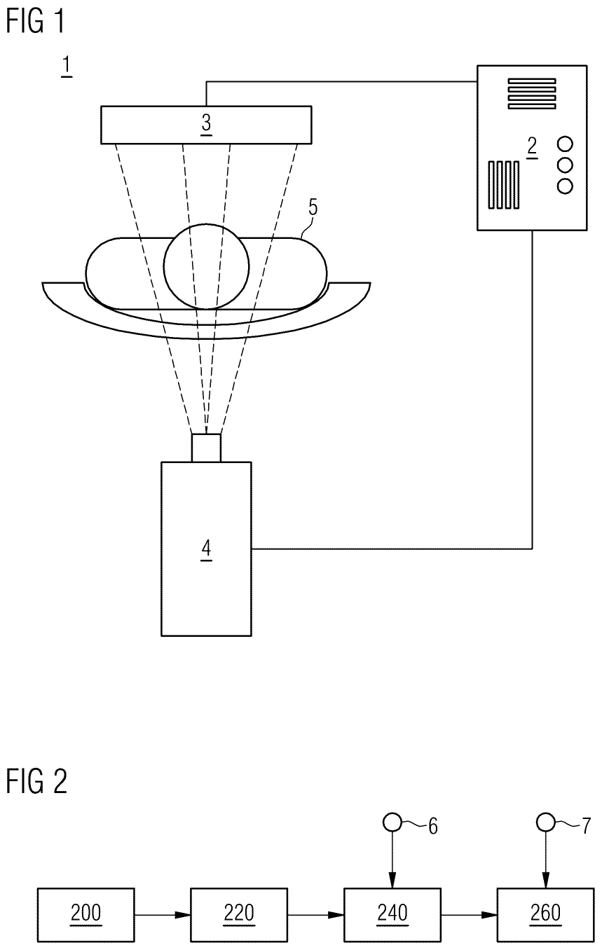

FIG. 1 shows a schematic representation of an example embodiment of an imaging system 1. The imaging system 1 has an imaging modality 3, 4 that in the present case is represented without limiting the generality as an X-ray imaging modality with an X-ray detector 3 and an X-ray source 4. The imaging system 1 has one embodiment of a data processing apparatus with at least one computing unit 2 that is configured to carry out a method of the present embodiments for assessing the effect of a medical instrument 8a, 8b, 8c, 8d, 13 during a vascular intervention (e.g., on a patient 5). FIG. 2 shows a schematic flowchart of an example embodiment of such a method of the present embodiments.

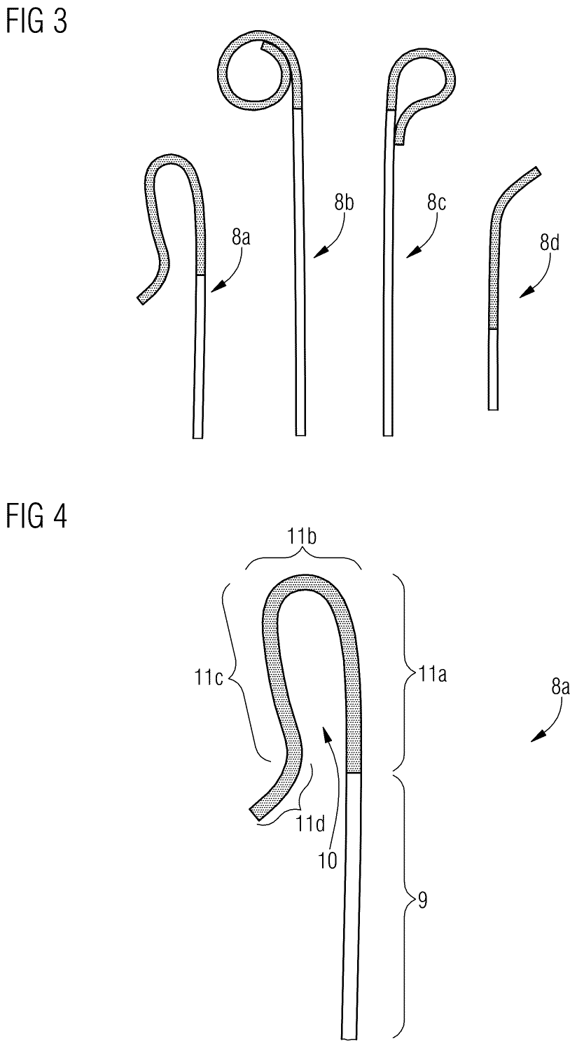

In act 200 of the method, the at least one computing unit 2 obtains a sequence of images that were generated by the imaging modality 3, 4. The sequence of images each show a vessel section 12 of a vascular tree of the patient 5. At least some of the images show a first medical instrument 8a, 8b, 8c, 8d, 13 arranged in the vessel section 12. The first medical instrument 8a, 8b, 8c, 8d, 13 may be, by way of example, a stent or a vessel catheter 8a, 8b, 8c, 8d, as schematically represented in FIG. 3 and FIG. 4, or a guide wire for minimally invasive interventions or the like.

In act 220, the at least one computing unit 2 ascertains a deformation of the vessel section 12 based on the sequence of images. The at least one computing unit 2 obtains first mechanical instrument properties 6 of the first medical instrument 8a, 8b, 8c, 8d, 13 and in act 240 determines mechanical vessel properties of the vessel section based on the first mechanical instrument properties 6 (e.g., a geometric shape and/or a diameter and/or a length and/or a rigidity and/or an elasticity and/or a surface lubricity of the first medical instrument 8a, 8b, 8c, 8d, 13) and based on the ascertained deformation of the vessel section 12.

The mechanical vessel properties may include, by way of example, a rigidity and/or an elasticity. The mechanical vessel properties may, for example, also vary locally (e.g., along the longitudinal direction of a main vessel).

The first medical instrument 8a, 8b, 8c, 8d, 13 may be selected, by way of example, based on a simulation model (e.g., a model that is based on the finite element method), with it being possible for the simulation model to form the basis, for example, of pre-operative planning data or a pre-operative CT reconstruction in order to initially assess the suitability of the first medical instrument 8a, 8b, 8c, 8d, 13.

The at least one computing unit 2 obtains second mechanical instrument properties 7 of a second medical instrument 8a, 8b, 8c, 8d, 13 different from the first medical instrument 8a, 8b, 8c, 8d, 13. In act 260, the at least one computing unit 2 assesses an expected deformation of the vessel section 12 during a potential vascular intervention on the vessel section 12 using the second medical instrument 8a, 8b, 8c, 8d, 13 based on the second mechanical instrument properties 7 and the mechanical vessel properties.

The deformation that arises owing to the use of the first medical instrument potentially differs from a deformation expected owing to the simulation model, by way of example, because the simulation model is not exact and/or because changes in the mechanical vessel properties have arisen since the pre-operative planning.

Based on the expected deformation of the vessel section owing to the use of the second medical instrument, which deformation is assessed using the method of the present embodiments, it is possible to decide whether the second medical instrument 8a, 8b, 8c, 8d, 13 is suitable for the potential vascular intervention (e.g., is more suitable than other medical instruments 8a, 8b, 8c, 8d, 13 or a third medical instrument).

It may also be provided that the simulation model is adapted to the actually ascertained circumstances based on the assessed, expected deformation and/or based on the ascertained mechanical vessel properties.

FIG. 3 shows examples of different vessel catheters 8a, 8b, 8c, 8d as possible medical instruments available for selection. The vessel catheters 8a, 8b, 8c, 8d shown each have a substantially straight shaft 9 and differently shaped tips 10, as is schematically represented in FIG. 4 for the vessel catheter 8a. The mechanical instrument properties 6, 7 may include, by way of example, corresponding shapes, sizes, elasticities, and/or surface lubricities, etc. for the tip 10 or possibly for different parts 11a, 11b, 11c, 11d of the tip 10.

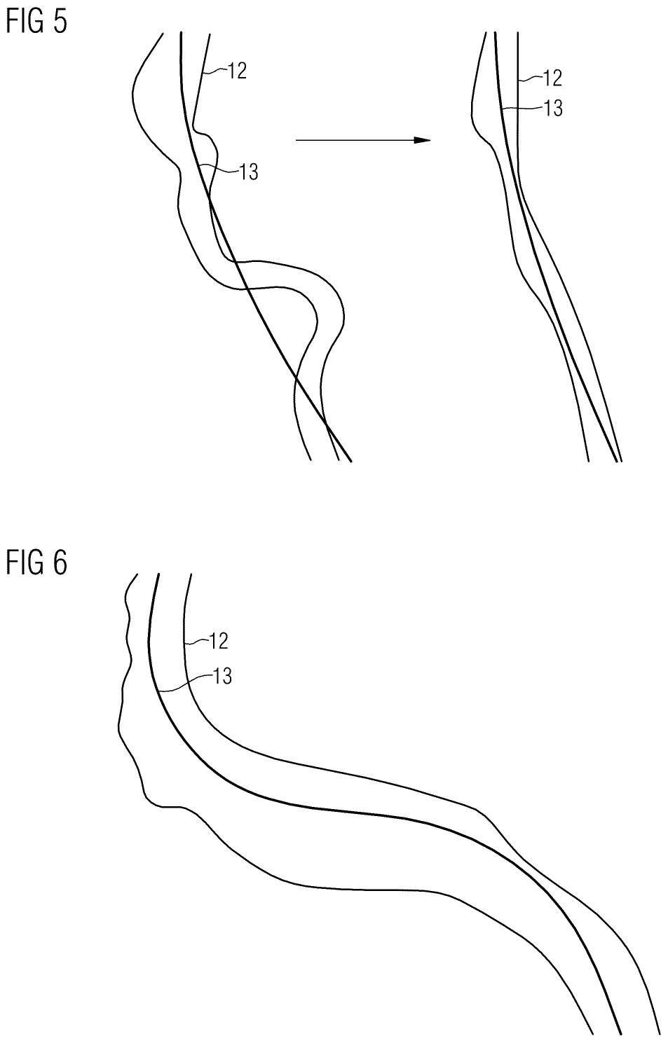

The left of FIG. 5 schematically shows a pre-operative image representation of the vessel section 12 and a medical instrument 13 that is overlaid on the image representation of the vessel section 12. When generating the image representation of the vessel section 12, the medical instrument 13 was therefore not actually introduced into the vessel section 12. The right of FIG. 5 schematically shows, as a result of an initial simulation, how the vessel section 12 would be deformed when the medical instrument 13 was introduced.

FIG. 6 schematically shows an image that shows the vessel section 12 and the medical instrument 13 after the medical instrument 13 was actually introduced into the vessel section 12. A deformation of the vessel section 12 that is different than the expected deformation in FIG. 5 results. The simulation model may be appropriately adapted based on the mechanical vessel properties that were derived from the actual deformation and the mechanical instrument properties of the medical instrument 13. The adjusted simulation model may then also be used, by way of example, to assess an expected deformation of the vessel section 12 by a different medical instrument 13. The corresponding medical instrument 13 may be evaluated, by way of example, by comparison with corresponding predefined limit values for the deformation, based on the thus assessed, expected deformation. For example, it is possible to decide whether the corresponding medical instrument 13 is suitable for, or how well suited it is to, the vascular intervention.

As described, in particular with reference to the figures, the present embodiments make it possible to assess more accurately the effect of a medical instrument on a vessel section (e.g., a corresponding deformation of the vessel section) during a vascular intervention.

In this connection, particular advantage is taken of the fact that if the mechanical instrument properties of an inserted medical instrument are known and an interaction of the medical instrument with the vessel section is ascertained intraoperatively (e.g., via angiography), it is possible to draw conclusions about the exact local mechanical vessel properties in the observed vessel section. In order to ascertain the mechanical vessel properties, values from the literature supplement or the like may also be used in some embodiments.

By way of example, a statement may thus be made about the extent to which the selected medical instrument fulfils the desired purpose or achieves an undesirable effect. Undesirable effects include, by way of example but not exclusively, excessive stretching, elongation, etc. of the vessel section, a deformation of the medical instrument beyond a load limit, etc. For the case where the medical instrument causes an undesirable effect, for example, the limiting property (e.g., the rigidity, the diameter, etc.) of the medical instrument may be ascertained. Further, by way of example, it is possible to predict exactly how further medical instruments with similarly known mechanical instrument properties will foreseeably behave in the vessel section. Different medical instruments (e.g., guide wires, stents, vessel catheters, etc.) may be evaluated correspondingly in accordance with their suitability for treatment and/or navigation in the vessel section.

The method of the present embodiments may be used for any vascular trees (e.g., also in neuroradiology or for liver interventions).

Independent of the grammatical term usage, individuals with male, female, or other gender identities are included within the term, and this includes the term “patient,” in particular.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for assessing the effect of a medical instrument during a vascular intervention, the method comprising:
obtaining a sequence of images, wherein the sequence of images map a vessel section and at least some images of the sequence of images map a first medical instrument arranged in the vessel section;
ascertaining a deformation of the vessel section based on the sequence of images;
obtaining first mechanical instrument properties of the first medical instrument;
determining mechanical vessel properties of the vessel section based on the first mechanical instrument properties and the deformation of the vessel section;
obtaining second mechanical instrument properties of a second medical instrument; and
assessing an expected deformation of the vessel section during a vascular intervention on the vessel section using the second medical instrument based on the second mechanical instrument properties and the mechanical vessel properties.

2. The method of claim 1, further comprising:
assessing a further expected deformation of the vessel section during a vascular intervention on the vessel section using the first medical instrument, using a predefined simulation model as a function of the first mechanical instrument properties and as a function of predefined initial vessel properties of the vessel section; and
selecting the first medical instrument from a predefined plurality of medical instruments as a function of the further expected deformation.

3. The method of claim 2, wherein:
the simulation model is updated as a function of the deformation and the further expected deformation;
the simulation model is updated as a function of the mechanical vessel properties and the initial vessel properties; or
a combination thereof.

4. The method of claim 3, wherein the expected deformation of the vessel section is assessed using the updated simulation model as a function of the second mechanical instrument properties and the mechanical vessel properties.

5. The method of claim 1, wherein the expected deformation of the vessel section during a vascular intervention on the vessel section using the second medical instrument is assessed using a predefined simulation model as a function of the second mechanical instrument properties and the mechanical vessel properties.

6. The method of claim 1, further comprising outputting an item of user information that proposes the second medical instrument for use during a vascular intervention on the vessel section as a function of the expected deformation.

7. The method of claim 1, wherein the first medical instrument is a first stent, the second medical instrument is a second stent, or the first medical instrument is the first stent and the second medical instrument is the second stent.

8. The method of claim 7, wherein
the first mechanical instrument properties include a geometric shape, a diameter, a length, a rigidity, an elasticity, a surface lubricity, or any combination thereof of the first stent;
the second mechanical instrument properties include a geometric shape, a diameter, a length, a rigidity, an elasticity, a surface lubricity, or any combination thereof of the second stent; or
a combination thereof.

9. The method of claim 1, wherein the first medical instrument is a first vessel catheter, the second medical instrument is a second vessel catheter, or the first medical instrument is the first vessel catheter and the second medical instrument is the second vessel catheter.

10. The method of claim 9, wherein:
the first mechanical instrument properties include a diameter, a geometric shape, a length, a rigidity, a surface lubricity of a tip or a shaft or part of the tip or the shaft, or any combination thereof of the first vessel catheter;
the second mechanical instrument properties include a diameter, a geometric shape, a length, a rigidity, a surface lubricity of a tip or a shaft or part of the tip or of the shaft, or any combination thereof of the second vessel catheter; or
a combination thereof.

11. The method of claim 1, wherein:
the sequence of images represents a movement of the first medical instrument;
the expected deformation is a deformation that is to be expected due to an intentional movement of the second medical instrument in the vessel section; or
a combination thereof.

12. The method of claim 1, wherein the sequence of images is a sequence of X-ray images.

13. A data processing apparatus comprising:
a processor configured to assess the effect of a medical instrument during a vascular intervention, the processor being configured to asses the effect of the medical instrument during the vascular intervention comprising the processor being configured to:
obtain a sequence of images, wherein the sequence of images map a vessel section, and at least some images of the sequence of images map a first medical instrument arranged in the vessel section;
ascertain a deformation of the vessel section based on the sequence of images;
obtain first mechanical instrument properties of the first medical instrument;
determine mechanical vessel properties of the vessel section based on the first mechanical instrument properties and the deformation of the vessel section;
obtain second mechanical instrument properties of a second medical instrument; and
assess an expected deformation of the vessel section during a vascular intervention on the vessel section using the second medical instrument based on the second mechanical instrument properties and the mechanical vessel properties.

14. An imaging system comprising:
a data processing apparatus comprising:
a processor configured to assess the effect of a medical instrument during a vascular intervention, the processor being configured to assess the effect of the medical instrument during the vascular intervention comprising the processor being configured to:
obtain a sequence of images, wherein the sequence of images map a vessel section, and at least some images of the sequence of images map a first medical instrument arranged in the vessel section;
ascertain a deformation of the vessel section based on the sequence of images;
obtain first mechanical instrument properties of the first medical instrument;
determine mechanical vessel properties of the vessel section based on the first mechanical instrument properties and the deformation of the vessel section;
obtain second mechanical instrument properties of a second medical instrument; and
assess an expected deformation of the vessel section during a vascular intervention on the vessel section using the second medical instrument based on the second mechanical instrument properties and the mechanical vessel properties; and
an imaging modality that is configured to generate the sequence of images.

15. In a non-transitory computer-readable storage medium that stores instructions executable by one or more processors to assess the effect of a medical instrument during a vascular intervention, the instructions comprising:
obtaining a sequence of images, wherein the sequence of images map a vessel section and at least some images of the sequence of images map a first medical instrument arranged in the vessel section;
ascertaining a deformation of the vessel section based on the sequence of images;
obtaining first mechanical instrument properties of the first medical instrument;
determining mechanical vessel properties of the vessel section based on the first mechanical instrument properties and the deformation of the vessel section;
obtaining second mechanical instrument properties of a second medical instrument; and
assessing an expected deformation of the vessel section during a vascular intervention on the vessel section using the second medical instrument based on the second mechanical instrument properties and the mechanical vessel properties.
